(19) United States
(12) Patent Application Publication
(10) Pub. No.: US 2021/0121093 A1
(43) Pub. Date: Apr. 29, 2021

(54) IMAGING TISSUE ANISOTROPY

(71) Applicant: KONINKLIJKE PHILIPS N.V., EINDHOVEN (NL)

(72) Inventor: Shlomo BEN-HAIM, EINDHOVEN (NL)

(21) Appl. No.: 17/257,655

(22) PCT Filed: Jul. 3, 2019
(86) PCT No.: PCT/EP2019/067914

§ 371 Date: Jan. 4, 2021

Related U.S. Application Data
(60) Provisional application No. 62/693,935, filed on Jul. 4, 2018.

(51) Int. Cl. A61B 5/0538; A61B 5/00; A61B 5/339

(52) U.S. Cl. CPC A61B 5/0538; A61B 5/6869; A61B 5/339; A61B 5/6876

(57) ABSTRACT

A method for measuring tissue conductance isotropy including measuring tissue conductance in a first direction, measuring tissue conductance in a second direction, and calculating tissue conductance isotropy based on the tissue conductance in the first direction and the tissue conductance in the second direction, wherein the second direction is not parallel to the first direction. A system for measuring tissue conductance isotropy including a catheter including a current source electrode, a plurality of induced voltage measuring electrodes, a signal processing unit for calculating tissue conductance isotropy based on tissue conductance measured in a first direction and tissue conductance measured in a second direction. Related apparatus, methods and computer program product are also described.

### FIELD AND BACKGROUND OF THE INVENTION

The present invention, in some embodiments thereof, relates to imaging electric conductance of a body, and, more particularly, but not exclusively, to imaging anisotropy of electric conductance in a body, and even more particularly but not exclusively, to imaging anisotropy of electric conductance in a body using measurements picked up by electrodes within a body.

The present invention, in some embodiments thereof, relates to measuring electric conductance of a tissue body, and, more particularly, but not exclusively, to measuring anisotropy of electric conductance in a tissue body, and even more particularly but not exclusively, to measuring anisotropy of electric conductance in a tissue body using signals picked up by electrodes within a body.

Cardiac electrical activation spreads from an active cardiac cell by electrical currents flowing from the activated cell (or group of cells) to adjacent (still quiescent) cell(s) and charges the cell(s) to reach a membrane voltage threshold, to activate an action potential (automatic response) which makes the adjacent cells “active”. The process is repeated and charges the cell(s) that are adjacent to the just-activated cell(s). Such is a method by which propagation of cardiac activation takes place.

Directionality of signal propagation—cells are sometimes structured in bundles with a specific longitudinal direction. Often the cells themselves have a longitudinal structure and are connected on to a neighbor cell by a low resistance structure, termed “intercalated disc”. Conversely, on a transverse direction, cells are often isolated one from the other by a fibrous tissue with high resistivity. When resistivity is measured along fibers a 10-times lower resistance is typically measured along the fibers than across the fibers. The directional property of conduction may be termed dispersion of conductivity.

Dispersion of conductivity in tissue causes a propagation speed of electric signals in a longitudinal direction to be faster (typically 3 times faster) than propagation speed in a transverse direction.

Additional background art includes:

An article by Ferrer, Ana & Sebastian, Rafael & Sánchez-Quintana, Damián & Rodriguez, Jose & Godoy, Eduardo & Martínez, Laura & Saiz, Javier. (2015). Titled “Detailed Anatomical and Electrophysiological Models of Human Atria and Torso for the Simulation of Atrial Activation”, published in PloS one. 10. e0141573. 10.1371/journal.pone.0141573.

An article by Christopher H. Fry, Rosaire P. Gray, Paramdeep S. Dhillon, Rita I. Jabr, Emmanuel Dupont, Pravina M. Patel, Nicholas S. Peters titled “Architectural Correlates of Myocardial Conduction Changes to the Topography of Cellular Coupling, Intracellular Conductance, and Action Potential Propagation with Hypertrophy in Guinea-Pig Ventricular Myocardium” published in Circulation: Arrhythmia and Electrophysiology. 2014; 7:1198-1204, Originally published Oct. 13, 2014.

An article by Bart Maesen, Stef Zeemering, Carlos Afonso, Jens Eckstein, Rebecca A. B. Burton, Arne van Hunnik, Daniel J. Stuckey, Damian Tyler, Jos Maessen, Vicente Grau, Sander Verheule, Peter Kohl, Ulrich Schotten, titled “Rearrangement of Atrial Bundle Architecture and Consequent Changes in Anisotropy of Conduction Constitute the 3-Dimensional Substrate for Atrial Fibrillation”, published in Circulation: Arrhythmia and Electrophysiology. 2013; 6:967-975, Originally published Oct. 15, 2013.

An article by Junaid A. B. Zaman, Nicholas S. Peters titled “The Rotor Revolution Conduction at the Eye of the Storm in Atrial Fibrillation”, published in Circulation: Arrhythmia and Electrophysiology. 2014; 7:1230-1236, originally published Dec. 16, 2014.

An article by Mélèze Hocini, Peter Loh, Siew Y. Ho, Damian Sanchez-Quintana, Bernard Thibault, Jacques M. T. de Bakker and Michiel J. Janse, titled “Anisotropic Conduction in the Triangle of Koch of Mammalian Hearts: Electrophysiologic and Anatomic Correlations”, published in Journal of the American College of Cardiology, Volume 31, Issue 3, March 1998.

The disclosures of all references mentioned above and throughout the present specification, as well as the disclosures of all references mentioned in those references, are hereby incorporated herein by reference.

### SUMMARY OF THE INVENTION

The present invention, in some embodiments thereof, relates to imaging electric conductance of a body, and, more particularly, but not exclusively, to imaging anisotropy of electric conductance in a body, and even more particularly but not exclusively, to imaging anisotropy of electric conductance in a body using measurements picked up by electrodes within a body.

The present invention, in some embodiments thereof, relates to measuring electric conductance of a tissue body, and, more particularly, but not exclusively, to measuring anisotropy of electric conductance in a tissue body, and even more particularly but not exclusively, to measuring anisotropy of electric conductance in a tissue body using signals picked up by electrodes within a body. Displaying and/or measuring a two-dimension or three-dimensional image of electric conductance of a tissue body, or of anisotropy of electric conductance in a tissue body potentially enables displaying: organs, muscle layers within organs, longitudinal directions of tissue/muscle and tissue/muscle layers, potentially illustrating diseases specific to layers, potentially illustrating location of conductance issues potentially related to atrial fibrillation (AF).

Displaying and/or measuring a two-dimension or three-dimensional image of electric conductance of a tissue body, or of anisotropy of electric conductance in a tissue body potentially enables diagnosing areas within the heart that have isotopic states, grade different isotropic states according to their isotropism, relate isotropism to probability for re-entrant formation and/or image tissue that is prone to create re-entry.

Displaying and/or measuring a two-dimension or three-dimensional image of electric conductance of a tissue body, or of anisotropy of electric conductance in a tissue body potentially enables diagnosing and/or treating of different arrhythmias, for example: diagnosing and/or treating of atrial fibrillation.

Some embodiments of the invention relate to methods to treat isotropic tissue to decrease its propensity to cause arrhythmias.

Displaying image of electric conductance of a tissue body may include imaging and/or mapping conductance directionality of isotropy values of tissue through its thickness.

In some embodiments measuring electric conductance of a body, or anisotropy of electric conductance in a body is performed by electrodes within a body.

According to an aspect of some embodiments of the present invention there is provided a method for measuring tissue conductance isotropy including measuring tissue conductance in a first direction, measuring tissue conductance in a second direction, and calculating tissue conductance isotropy based on the tissue conductance in the first direction and the tissue conductance in the second direction, wherein the second direction is not parallel to the first direction.

According to some embodiments of the invention, values of tissue conductance in the first direction and in the second direction are calculated as tissue conductance in a longitudinal direction CL and in a perpendicular transverse direction CT.

According to some embodiments of the invention, the values of tissue conductance in a longitudinal direction CL is determined in a direction of maximum conductance.

According to some embodiments of the invention, the values of tissue conductance in a transverse direction CT is determined in a direction of minimum conductance.

According to some embodiments of the invention, the measuring tissue conductance in the first direction is performed by providing a current source at a source location and measuring induced voltage at a first measuring location.

According to some embodiments of the invention, the measuring tissue conductance in the second direction is performed by providing the current source at the source location and measuring induced voltage at a second measuring location.

According to some embodiments of the invention, the measuring tissue conductance in the first direction and the measuring tissue conductance in the second direction are performed simultaneously.

According to some embodiments of the invention, the current source is provided by an electrode implanted in tissue.

According to some embodiments of the invention, the measuring tissue conductance in the first direction and the measuring tissue conductance in the second direction is performed by a measuring electrode on a same implanted electrode as the current source.

According to some embodiments of the invention, the measuring tissue conductance in the first direction and the measuring tissue conductance in the second direction is performed by a measuring electrode on a same implanted electrode as the current source.

According to some embodiments of the invention, the current source is provided by an electrode on a catheter.

According to some embodiments of the invention, the measuring tissue conductance in the first direction is performed by a measuring electrode provided on a catheter.

According to some embodiments of the invention, the measuring tissue conductance in the second direction is performed by a measuring electrode provided on a same catheter as the current source.

According to some embodiments of the invention, the measuring tissue conductance in the second direction is performed by a measuring electrode provided on a same catheter as the current source.

According to some embodiments of the invention, the catheter is placed next to the tissue being measured.

According to some embodiments of the invention, the catheter is within a body cavity during the measurement.

According to some embodiments of the invention, the catheter is within a blood vessel during the measurement.

According to some embodiments of the invention, the catheter is within a heart during the measurement.

According to some embodiments of the invention, tissue conductance is measured in more than two directions at a same source location.

According to some embodiments of the invention, tissue conductance is measured in more than two directions simultaneously.

According to some embodiments of the invention, the catheter is translated along the tissue, additional conductance measurements are performed, and further including providing locations of the measurements.

According to some embodiments of the invention, same electrodes are used to measure conductance and to provide data for providing the locations.

According to some embodiments of the invention, further including producing a map of tissue conductance isotropy based, at least in part, on the locations.

According to some embodiments of the invention, the map is selected from a group consisting of a one-dimensional map, a two-dimensional map, and a three-dimensional map.

According to some embodiments of the invention, the map displays different tissue conductance isotropy using different colors.

According to some embodiments of the invention, the calculating tissue conductance isotropy is performed for a same location at different times, and a change in tissue conductance isotropy is calculated.

According to some embodiments of the invention, the calculating tissue conductance isotropy is performed at different times during one cardiac cycle.

According to some embodiments of the invention, the tissue conductance isotropy is combined with ECG data.

According to some embodiments of the invention, the map is produced for a same location at different times, and a map of change in tissue conductance isotropy is calculated.

According to some embodiments of the invention, the map of change is displayed in color based on an amount of change.

According to an aspect of some embodiments of the present invention there is provided a system for measuring tissue conductance isotropy including a catheter including a current source electrode, a plurality of induced voltage measuring electrodes, a signal processing unit for calculating tissue conductance isotropy based on tissue conductance measured in a first direction and tissue conductance measured in a second direction.

According to some embodiments of the invention, the current source electrodes and the plurality of induced voltage measuring electrodes are included in a catheter.

According to some embodiments of the invention, at least one electrode is a directional electrode.

According to some embodiments of the invention, at least one electrode includes a cylindrical electrode area.

According to some embodiments of the invention, the signal processing unit is configured to transform values of tissue conductance in the first direction and in the second direction to tissue conductance in a longitudinal direction CL and in a perpendicular transverse direction CT.

According to some embodiments of the invention, the signal processing unit is configured to calculate tissue conductance isotropy based on a ratio of the tissue conductance values in two different directions.

According to some embodiments of the invention, the signal processing unit further includes a connection for transmitting values to an external receiving unit.

According to some embodiments of the invention, the signal processing unit further includes a connection for transmitting values to an external display unit.

According to some embodiments of the invention, the current source electric contact and the plurality of induced voltage measuring electric contact are included in an implantable electrode.

According to some embodiments of the invention, the signal processing unit is included in an implantable cardiac pacemaker.

According to an aspect of some embodiments of the present invention there is provided a system for measuring tissue conductance isotropy including contact signal transmitting means, contact signal receiving means, remote signal receiving means, a signal processing unit, and a display unit, wherein the signal processing unit is configured to calculate an impedance between the contact transmitting and receiving means, adjusting for transmitting the impedance to the receiving means.

According to an aspect of some embodiments of the present invention there is provided a method for calculating P_{re-entry} including measuring tissue conductance isotropy, and calculating Pre-entry based on the measured tissue conductance isotropy.

According to some embodiments of the invention, further including displaying P_{re-entry}.

According to an aspect of some embodiments of the present invention there is provided a method for mapping tissue conductance isotropy including associating a location on tissue with a co-located tissue conductance isotropy.

According to some embodiments of the invention, further including displaying a mapping of the tissue conductance isotropy to the location on the tissue.

According to an aspect of some embodiments of the present invention there is provided a method for mapping tissue conductance isotropy including receiving measurements of crossing electromagnetic fields using two sensors carried on an intra-body catheter at a known distance from each other, the measuring being carried out with the catheter at multiple locations in the body cavity, and reconstructing a shape of the body cavity, based on the received measurements, measuring tissue conductance isotropy, based on the received measurements, and associating a location on tissue with a co-located tissue conductance isotropy.

According to some embodiments of the invention, further including displaying a mapping of the tissue conductance isotropy to the location on the tissue.

According to an aspect of some embodiments of the present invention there is provided a system for measuring tissue conductance isotropy including means for measuring tissue conductance in a first direction, means for measuring tissue conductance in a second direction, and means for calculating tissue conductance isotropy based on the tissue conductance in the first direction and the tissue conductance in the second direction.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as displaying conductance or anisotropy of conductance, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are prior art graphs showing between intracellular resistivity (Ri), gap junctional (Rj) resistance, and conduction velocity in human and guinea pig myocardium;

FIG. 1C is a simplified illustration of some interconnected cells;

FIG. 2 is a component in a system for acquiring an image of electric conductance of a tissue body according to some embodiments of the invention;

FIG. 3 is a simplified illustration of a system for acquiring image of electric conductance of a tissue body according to some embodiments of the invention;

FIG. 4A is a simplified illustration of conducting fibers arranged in layers;

FIG. 4B is a simplified illustration of conducting fibers arranged in layers;

FIG. 5 is a simplified illustration of a model of resistance along and between conducting fibers, according to an example embodiment of the invention;

FIG. 6 is a simplified illustration of a model of resistance along and between conducting fibers, according to an example embodiment of the invention;

FIG. 7 is a simplified block diagram illustration of a system according to some embodiments of the invention;

FIG. 8 is a simplified block diagram illustration of a system according to some embodiments of the invention;

FIG. 9A is a simplified flow chart illustration of a method for measuring tissue conductance isotropy according to some embodiments of the invention; and

FIG. 9B is a simplified flow chart illustration of a method for mapping tissue conductance isotropy according to some embodiments of the invention.

### DESCRIPTION OF SPECIFIC EMBODIMENTS OF THE INVENTION

The present invention, in some embodiments thereof, relates to imaging electric conductance of a body, and, more particularly, but not exclusively, to imaging anisotropy of electric conductance in a body, and even more particularly but not exclusively, to imaging anisotropy of electric conductance in a body using measurements picked up by electrodes within a body.

The present invention, in some embodiments thereof, relates to measuring electric conductance of a tissue body, and, more particularly, but not exclusively, to measuring anisotropy of electric conductance in a tissue body, and even more particularly but not exclusively, to measuring anisotropy of electric conductance in a tissue body using signals picked up by electrodes within a body.

In disease states, dispersion of conductivity often changes from the normal state. The gap junctions (the connection between cells is termed gap junctions) are sometimes lost, and resistance in the longitudinal direction sometimes increases. In some cases there may be infiltration of fibrous tissue that increases anisotropy or non-homogeneity of conduction.

Arrhythmia, such as atrial fibrillation and/or ventricular fibrillation can be related to presence of a changed dispersion of conductivity, for example a decreased dispersion of conductivity.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Overview of Some Aspects of the Invention

An aspect of some embodiments of the invention relates to measuring anisotropy of tissue conductance.

In some embodiments a model or mapping of tissue conductance in various directions, e.g. transverse and longitudinal relative to a catheter, is produced.

The terms “conductance” and “electric conductance” in all their grammatical forms are used in the specification and claims to mean conductance-per-volume of matter, that is, a value characterizing matter such as tissue, normalized to a unit of length and to a unit of cross-section.

The terms “impedance” and “resistance” in all their grammatical forms are used in the specification and claims to mean an inverse of “conductance” and its grammatical forms, that is, 1/“conductance”.

The term conductance anisotropy in all its grammatical forms is used in the specification and claims to mean a value representing different conductance in different directions.

In some embodiments a model of anisotropy of tissue conductance is produced.

In some embodiments the model is produced by measuring the tissue conductance in a single body. In some embodiments the model is produced by measuring the tissue conductance in multiple bodies and producing the model based on typical values for a typical body.

In some embodiments, a measurement of tissue conductance value or conductance anisotropy value serves as a value used to determine location of the measuring catheter in the body by comparing the value to values in the model. In some embodiments, the value serves for determining location on its own. In some embodiments, the value is used in combination with additional measured parameters, e.g., additional electrical parameters such as described in PCT Patent Application IB 2018/050192 of Dichterman et al., titled “Systems And Methods For Reconstruction Of Intra-Body Electrical Readings To Anatomical Structure”, or additional parameters such as distance of insertion of the catheter or of an electrode inserted through the catheter.

Exemplary methods for estimating and/or measuring and/or evaluating impedance based on measurements made at catheter electrodes (intra-body electrodes) are described in U.S. Provisional Patent Application No. 62/667,530 titled “MEASURING ELECTRICAL IMPEDANCE, CONTACT FORCE AND TISSUE PROPERTIES”. Such methods may be used for measuring electric conductance of a tissue body and/or for measuring anisotropy of electric conductance in a tissue body.

An aspect of some embodiments of the invention relates to mapping anisotropy of tissue conductance in a body tissue and producing an image and/or a mathematical model of the conductance anisotropy of the body tissue using the conductance anisotropy values.

The model may be 1, 2, or 3 dimensional.

A 1-dimensional model of conductance anisotropy may be, by way of a non-limiting example, a model or map of conductance anisotropy along a blood vessel, optionally as measured by a catheter along the vessel.

A 2-dimensional model of conductance anisotropy may be, by way of a non-limiting example, a model or map of conductance anisotropy of a surface of a body and/or of a surface of a body cavity, optionally as measured by a catheter passing near and/or in contact with the surface. By way of a non-limiting example, a 2-d model or map can be made of an inside surface of a heart ventricle and/or atrium by a catheter traveling within the heart ventricle and/or atrium.

A 3-dimensional model of conductance anisotropy may be, by way of a non-limiting example, a model or map of conductance anisotropy of a volume of a body and/or of a body organ, optionally as measured by a catheter passing near and/or within and/or in contact with the organ. By way of a non-limiting example, a 3-d model or map can be made of an inside surface of heart muscle by a catheter traveling within the heart and/or by a catheter traveling along a blood vessel near the heart or on a surface of the heart.

In some embodiments electric conductance and/or anisotropy of electric conductance of a body is optionally displayed.

In some embodiments, since different organs possess potentially different conductance anisotropy properties, the display uses a different display property, for example a different color, to display anisotropy values.

In some embodiments, the display displays different organs in different colors, at least partly based on the different anisotropy.

In some embodiments, the display displays a diseased organ or a diseased part of an organ, in a different color than a healthy organ or a healthy part of an organ, at least partly based on the different anisotropy.

In some embodiments, the display displays different tissue layers using different colors, at least partly based on the different anisotropy.

In some embodiments, the display displays different muscle layers using different colors, at least partly based on the different anisotropy.

An aspect of some embodiments of the invention relates to measuring anisotropy of tissue conductance to determine a location of a catheter inside a body.

### Identifying Changes

An aspect of some embodiments of the invention relates to identifying changes in anisotropy of tissue conductance in a body.

In some embodiments, a time span of identifying changes may span a relatively long time, such as 1-24 hours, 1-7 days, 1-4 or 5 weeks, 1-12 months, and 1-100 years. By way of a non-limiting example conductance anisotropy a body is optionally mapped at a first time T1, and at a second time T2, and changes between the mappings are calculated. In some embodiments a change in conductance anisotropy indicates a disease. In some embodiments specific locations in a body are optionally monitored for changes in conductance anisotropy, targeting specific diseases. By way of a non-limiting example, monitoring a heart potentially enables detecting a re-entrant activation of an activation wave front, and/or atrial fibrillation, potentially even in early stages.

In some embodiments a change is identified in a first specific area in a mapping, based on the change in the first area being significantly higher than a change in another, second area of the mapping.

In some embodiments, a time span of identifying changes may span a relatively short time, such as portions of a second, seconds, or minutes. By way of a non-limiting example conductance anisotropy may be measured more than once within a heartbeat. By way of a non-limiting example conductance anisotropy may be measured at different positions along a heartbeat sequence.

In some embodiments, identifying changes may be performed between mappings when muscle is under stress and when muscle is at rest. By way of a non-limiting example, conductance anisotropy may be measured in or near a heart during drug-induced or exercise-induce stress and during rest.

### Identifying Diseases

An aspect of some embodiments of the invention relates to determining a disease and/or advance in disease status, and/or a change in disease status, based on identifying changes in anisotropy of tissue conductance in a body.

In some embodiments, tracking and/or identifying atrial fibrillation in a patient is performed by identifying changes in anisotropy of tissue conductance in the patient's heart.

In some embodiments, such as, by way of a non-limiting example, tracking atrial fibrillation in a patient, a map of cardiac anisotropy is optionally made in an area of the body which includes a conduction system of the heart. Changes in the mapping are optionally tracked over time.

In some embodiments the mapping and/or display of anisotropy of electric conductance in a body is used to display and/or identify diseases such as, by way of some non-limiting examples, torn muscle, atrial fibrillation (AF), torn ligaments, torn uterus and hernia.

### Intra-Body Electrodes

An aspect of some embodiments of the invention relates to mapping conductance anisotropy using electrodes inside a body (referred to herein as intra-body electrodes).

In some embodiments the intra-body electrodes are inserted into a body through a catheter (e.g., the intra-body electrodes may be part of the catheter or otherwise attached to the catheter) and guided to an area of interest for mapping (e.g., for conductance anisotropy mapping). By way of a non-limiting example the area of interest includes a heart.

In some embodiments the intra-body electrodes are implanted in a body (referred herein as implanted electrode). By way of a non-limiting example the intra-body electrodes may be electrodes of a cardiac pacemaker. In some embodiments software for measuring conductance anisotropy is optionally included in a cardiac pacemaker. Optionally, the cardiac pacemaker is configured to transmit measurements and/or conductance anisotropy values to an external receiver.

In some embodiments an implanted unit for measuring conductance anisotropy optionally transmits measurements and/or conductance anisotropy values to an external receiver.

### Calculating Conductance Anisotropy

In some embodiments, electrical conductance values are measured (referred herein as conductance measurements) between electrodes, and the electrodes are moved inside a body.

In some embodiments, locations of the electrodes are known, and as the electrodes move, their locations are optionally recorded and/or transmitted to a calculation unit. In some embodiments, the locations of the electrodes are calculated based on measurements received by the electrodes, for example as described in the above-mentioned PCT Patent Application IB 2018/050192.

In some embodiments, a direction of maximum values of the conductance measurements is optionally determined to be a longitudinal direction.

In some embodiments some or all of the conductance measurements are optionally projected on the longitudinal direction and longitudinal conductance values are calculated.

In some embodiments a transverse direction is optionally determined. In some embodiments the transverse direction is optionally perpendicular to the longitudinal direction.

In some embodiments some or all of the conductance measurements are optionally projected on the transverse direction and transverse conductance values are calculated.

In some embodiments conductance is measured knowing a direction of the measuring electrodes or device relative to a longitudinal direction or a transverse direction of tissue, and conductance and/or conductance anisotropy are calculated based on the knowledge.

In some embodiments conductance is measured as described in described in U.S. Provisional Patent Application No. 62/667,530 titled “MEASURING ELECTRICAL IMPEDANCE, CONTACT FORCE AND TISSUE PROPERTIES”.

### Electrodes

In some embodiments, electrical conductance values are measured between two electrodes.

In some embodiments, electrical conductance values are measured between more than two electrodes.

In some embodiments, electrical conductance values are measured between intra-body electrodes.

In some embodiments, electrical conductance values are measured between one or more intra-body electrode(s) and one or more electrodes external to a body.

In some embodiments, electrical conductance values are measured between electrodes arranged in a specific geometry. By way of a non-limiting example two electrodes optionally define a straight line between them—and the straight line may optionally be along a catheter direction, across the catheter direction, or diagonal relative to the catheter direction. In some embodiments three or more electrodes are optionally geometrically arranged so that there are perpendicular paths between at least two pairs of electrodes.

In some embodiments the electrodes are omni-directional electrodes.

In some embodiments a catheter includes an electrode at its tip and one or more additional electrodes as ring electrodes along its length.

### Methods of Measurement

An aspect of some embodiments of the invention relates to methods of measuring conductance anisotropy.

In some embodiments one or more electrodes are in contact with tissue for which conductance is being measured.

In some embodiments one or all electrodes are not in contact with the tissue for which conductance is being measured.

An aspect of some embodiments of the invention includes mapping conductivity of the tissue. By way of a non-limiting example, a catheter is inserted into a body, which injects a current, for example from an electrode at a location C1. An electric field is optionally measured by an electrode or electrodes at one or more locations, for example C2, C3 and C4. The one or more locations (for example C2, C3 and C4) may refer to measurements by additional electrodes provided on the catheter at locations C2, C3 and C4. Optionally, the one or more locations may refer to measurements by single electrodes provided on the catheter as the electrode moves to locations C2, C3 and C4. In some embodiments a map of conductivity is displayed, for example a map of conductivity of a wall of a heart chamber.

In some embodiments data from a method such as described in PCT Patent Application IB 2018/050192 of Dichterman et al., titled “Systems And Methods For Reconstruction Of Intra-Body Electrical Readings To Anatomical Structure” is optionally used to determine location of a conductance measurement and/or of the conductance measurement device and/or the conductance measurement electrode(s),

In some embodiments a user optionally inputs location of a catheter, conductance measurement device, or electrodes.

### Identifying Disease Based on Conductance Anisotropy

In some embodiments changes of conductance anisotropy, from a normal state as defined in the literature and/or from a previous measurement, model or state of a same patient, are optionally used to detect a disease.

By way of a non-limiting example, conductance isotropy lower than what is defined as normal can potentially indicate a disease.

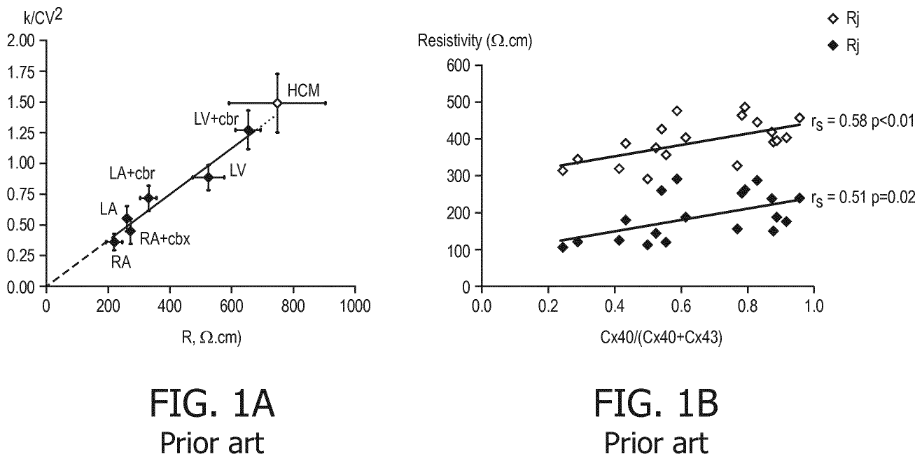

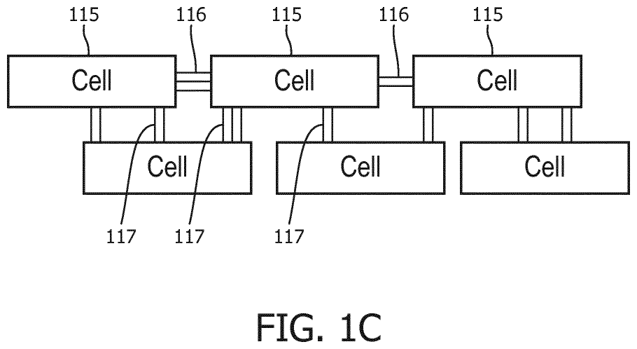

Reference is now made to FIGS. 1A and 1B, which are prior art graphs showing between intracellular resistivity (Ri), gap junctional (Rj) resistance, and conduction velocity in human and guinea pig myocardium. FIG. 1A shows correlation between intracellular resistivity (Rᵢ), gap junctional (Rⱼ) resistance, and conduction velocity in human and guinea pig myocardium. Left atrium (LA), right atrium (RA), left ventricle (LV), and hypertrophic cardiomyopathy (HCM) samples with or without the presence of gap junctional de-coupler carbenoxolone (CBX). FIG. 1B shows relative Cx ratio (Cx40/Cx40+Cx43) was significantly associated with Rᵢ/Rⱼ in human atrial trabeculae. (Reprinted from the above-mentioned article by Dhillon with permission of the publisher. Copyright© 2014 (panel A), 2013 (panel B), Wolters Kluwer Health.

The above-mentioned article titled “Anisotropic Conduction in the Triangle of Koch of Mammalian Hearts: Electrophysiologic and Anatomic Correlations” describes a disease other than AF which is believed to be caused by a change in anisotropic conduction.

### Further Discussion

Body tissue has electrical activity of the cells that form the tissue. The electrical activity can take form of action potential in some of the tissues.

Action potential typically has two phases: an excitable phase and a refractory phase.

Cells can be excited when they are in the excitable phase by an excitation current that is injected into the cell. Such current can be generated when an excitable cell is adjacent to an active and refractory cell.

In some tissues, electrical activity propagates from one cell to its neighbor producing a wave front of activation.

Some body tissue has a micro arrangement made of cells that are arranged in fibers. The fibers have a narrow width and a long length.

In some tissue an arrangement of cells is according to a direction of a longitudinal direction of a single cell. In some body tissues the cells are connected one to the other with special junctions. Some junctions provide a lower resistance for electrical current to pass from one cell to its neighbor cell.

Reference is now made to FIG. 1C, which is a simplified illustration of some interconnected cells.

FIG. 1C shows elongate cells 115 and inter-cell connections or junctions 116 117. Some of the junctions are longitudinal junctions 116, and some of the junctions are transverse junctions 117.

In some tissue an electric connection between cells is in form of a GAP junction. Some GAP junctions contain connecting protein-. Such protein lowers resistance of the gap junctions.

A density of GAP junctions may be different between longitudinal cell connections and transverse cell connections.

Such different GAP junction densities create a preferred path for electrical charge to pass between adjacent cells. Typical cardiac tissue, for example, has a 1:10 ratio between the longitudinal resistance between cells and the transverse resistance between cells.

The different resistances provide a faster charge time in the longitudinal direction, so that a propagation velocity of electrical activation is faster in the longitudinal direction.

By way of a non-limiting example, in normal cardiac tissue, longitudinal conduction velocity is faster (for example: three times faster) than the conduction velocity in the transverse direction.

It is noted that in the example of the heart, presence of anisotropy between conduction velocities creates a typical oval shaped activation wave front in normal cardiac tissue.

Cardiac arrhythmias can have multiple causation mechanisms.

One common mechanism is a re-entrant activation of the activation wave front.

In such a case an activation wave front can create a closed “self-activating” circuit, that can be constant or variable, but in both cases a circuit will activate itself; different from a normal conduction that propagates in one direction and “dies” each time the activation front reaches tissue boundary, and a next activation front of a normal heart tissue is generated from a normal sinus node pace maker.

In some disease states, there are conditions that alter the normal anisotropy and increase likelihood of re-entry arrhythmia formation.

Some disease states include alteration of tissue fiber micro structure, and changes in the connection between cells.

One result of disease can be creation of a more isotopic tissue where there is less of a preferred direction, or no single preferred direction, such that activation can propagate backward after certain distance, potentially creating conditions which cause a re-entry circuit.

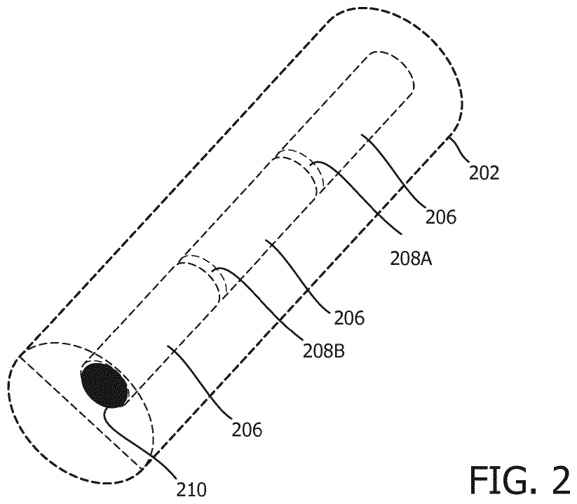

Reference is now made to FIG. 2, which is a component in a system for acquiring an image of electric conductance of a tissue body according to an example embodiment of the invention.

FIG. 2 shows a catheter 206 that may be used for measuring and/or imaging electric conductance of a tissue body.

In the following detailed description, the term catheter may refer to any physical carrier of one or more electrodes for insertion of the one or more electrodes into a living body—for example: endoscope, colonoscope, enteral feeding tube, stent, graft, etc . . . , which may be used for measuring and/or imaging electric conductance of a tissue body, for example: to identify changes in anisotropy of tissue conductance in a body.

In some embodiments, catheter 206 is inserted into a body lumen, for example a blood vessel 202.

In some embodiments, catheter 206 optionally includes two or more electrodes such as the electrodes 208A 208B shown in FIG. 2. The present invention is not limited to the use of two electrodes, additional electrodes may be used, e.g., 4, 6, 10, 15, 20 or 40. The electrodes may of different shape, size, or material.

At least one of electrodes 208A 208B may act as transmitting electrode and/or as receiving electrode (may function as a sensor).

In some embodiments, at least one of the electrodes 208A 208B is a ring electrode. In some embodiments, all of the electrodes 208A 208B are ring electrodes.

In some embodiments, the catheter 206 optionally includes an electrode 210 at the tip of the catheter 206. In some embodiments, electrodes 208A 208B (and optionally 210) are contact electrodes.

In some embodiments at least two of electrodes with a known distance between them are optionally brought in contact with the body tissue. The distance may be known to the system and may be used by one or more methods of the system, for example as in above-mentioned PCT Patent Application IB 2018/050192.

In some embodiments a remote or ground (reference) receiving electrode is included on catheter 206, e.g., for measuring current or voltage between two electrodes (one being the ground electrode).

In some embodiments the ground electrode is at a tip of the catheter, at a location of the electrode 210 of FIG. 2.

In some embodiments at least one current source is connected to at least one of the electrodes 208A 208B.

In some embodiments at least one of electrodes 208A and 208B is activated as a measuring electrode simultaneously with activation of at least one transmitting electrode.

In some embodiments the measuring electrode measures an induced voltage on the measuring electrode due to signal transmitted by the transmitting electrode.

In some embodiments the measuring is optionally a measuring of induced voltage on a remote measuring electrode due to signal transmitted by the transmitting electrode.

In some embodiments, the electrodes 208A 208B 210 and/or electrodes not shown are optionally arranged in a geometric configuration to measure conductance in two different not-parallel directions.

In some embodiments conductance measurement is optionally made between one set of electrodes whose distance apart is greater than another set of electrodes. The conductance measured by a pair of electrodes includes a longitudinal portion, along a direction between the electrodes, and a transverse portion, perpendicular to the longitudinal direction. The set of electrodes whose distance apart is greater measures conductance with a greater longitudinal portion the electrodes whose distance apart is smaller, potentially enabling to calculate the longitudinal portion and the transverse portion of the conductance, even using a set of electrodes arranged in a straight line.

In some embodiments the conductance is optionally measured between electrodes on a catheter, optionally inside a body, and one or more electrodes on a surface of the body.

In some embodiments a same sensor is optionally used for measuring conductance and for mapping location.

In some embodiments a same sensor is optionally used for measuring conductance and for providing data by which location is determined. In some embodiments the method for providing location is optionally as described in above-mentioned PCT Patent Application IB 2018/050192.

In some embodiments, at least one of the electrodes provided on catheter 206 function as a sensor.

In some embodiments conductance is measured within a duration of a single heartbeat.

In some embodiment's conductance is measured more than once within a duration of a single heartbeat.

In some embodiments conductance is measured and position relative to heart muscle is optionally determined. In some embodiments the position is optionally determined using a method such as described in above-mentioned PCT Patent Application IB 2018/050192.

In some embodiments conductance data is optionally combined with one or more of: ECG data; a cardiac activation time; additional data sensed by the catheter, additional data otherwise provided, such as, for example, imaging data.

In some embodiments location of the catheter is optionally provided by a non-impedance and/or non-dielectric method, such as, by way of a non-limiting example, magnetic-based imaging.

In some embodiments location of the catheter is optionally provided by an imaging method such as roentgen, x-ray, ultrasound.

In some embodiments one or more of the electrode(s) is directional.

In some embodiments one or more of the electrode(s) is omni-directional.

In some embodiments one or more of the electrode(s) has a cylindrical surface area.

In some embodiments one or more of the electrode(s) is a ring electrode.

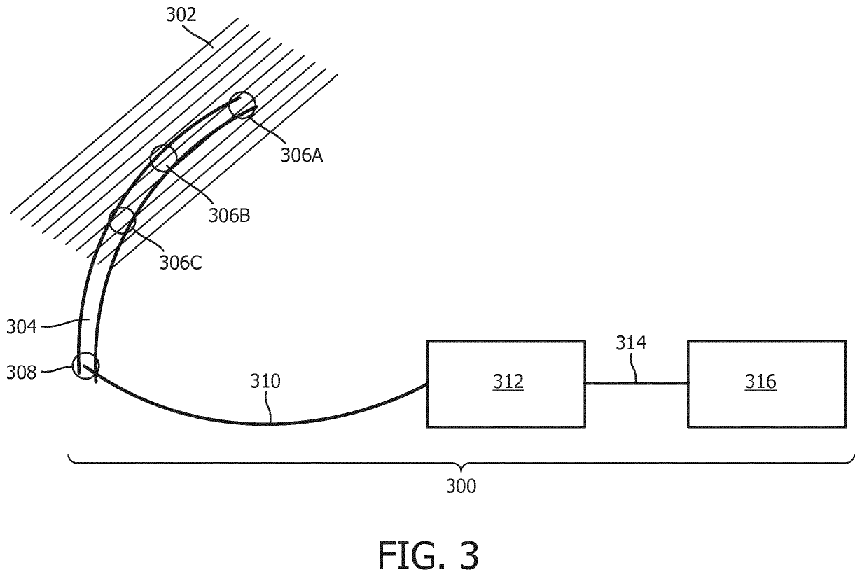

Reference is now made to FIG. 3, which is a simplified illustration of a system 300 for acquiring an image of electric conductance of a tissue body according to an example embodiment of the invention.

The system 300 may be used for measuring and/or imaging electric conductance of a tissue body, for example: to identify changes in anisotropy of tissue conductance in a body.

FIG. 3 shows a system 300 including a catheter 304 which includes electrodes 306A 306B 306C in, on or next to tissue 302, connected, optionally via an exit of the catheter 308, by a signal communication connection 310 to a signal processing unit 312. Catheter 304 may be, in some embodiments, identical or substantially identical to catheter 206 of FIG. 2.

In some embodiments, signal communication connection 310 is a signal transmission cable. In some embodiments, signal communication connection 310 is a wireless signal.

In some embodiments, signal processing unit 312 optionally includes a further connection 314 to an output unit 316. In some embodiments, output unit 316 is a display. In some embodiments, output unit 316 is a communication unit for sending results of the mapping and/or measuring of conductance isotropy to some external unit such as a display device or a storage device or a medical database.

In some embodiments signals from transmitting and the receiving electrodes are conveyed to signal processing unit 312.

In some embodiments, signal processing unit 312 optionally calculates a contact inter-electrode impedance, for example: as described in U.S. Provisional Patent Application No. 62/667,530.

In some embodiments, signal processing unit 312 optionally calculates contact to remote inter-electrode impedance.

In some embodiments, signal processing unit 312 optionally calculates tissue impedance between two contact sites.

In some embodiments, signal processing unit 312 optionally calculates a map electric conductance of a tissue body, e.g., by connecting the tissue impedance measured and its respective location. Optionally, the measured electric conductance at one location is registered such location on an anatomical image of such location.

In some embodiments, signal processing unit 312 optionally calculates tissue conductance anisotropy, e.g., based on such mapping or tissue impedance measurements.

In some embodiments, signal processing unit 312 optionally calculates changes in anisotropy of tissue conductance in a body, e.g., based on such mapping.

In some embodiments, system 300 optionally includes a data display for showing tissue impedance. In some embodiments, system 300 optionally includes a data display for displaying electric conductance of a tissue body and/or changes in anisotropy of tissue conductance in a body. In some embodiments, system 300 optionally includes a data display for displaying electric conductance of a tissue body on an anatomical image of such tissue body.

In some embodiments, system 300 optionally includes a multi-electrode catheter (such as: catheter 304 or 206). System 300 optionally includes more than one catheter.

In some embodiments, the multi-electrode catheter enables measuring multiple tissue impedances with the catheter, when the catheter is placed at a constant location (e.g., to minimize catheter movement within patient body). Additionally or alternatively, multiple tissue impedances may be measured by moving a catheter within patient body.

In some embodiments, the multi-electrode catheter enables measuring multiple tissue impedances simultaneously.

In some embodiments, system 300 is optionally configured to calculate a composite tissue conductance in two dimensions (2D), based. At least in part, on knowing a 2D arrangement of the electrodes.

In some embodiments, system 300 is optionally configured to determine multiple tissue conductances at multiple depths within a tissue.

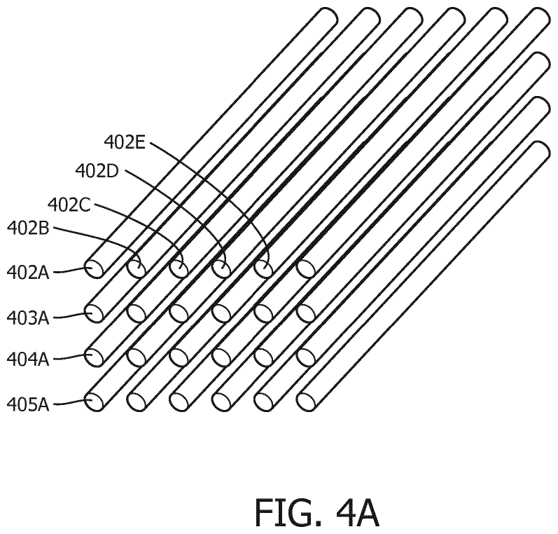

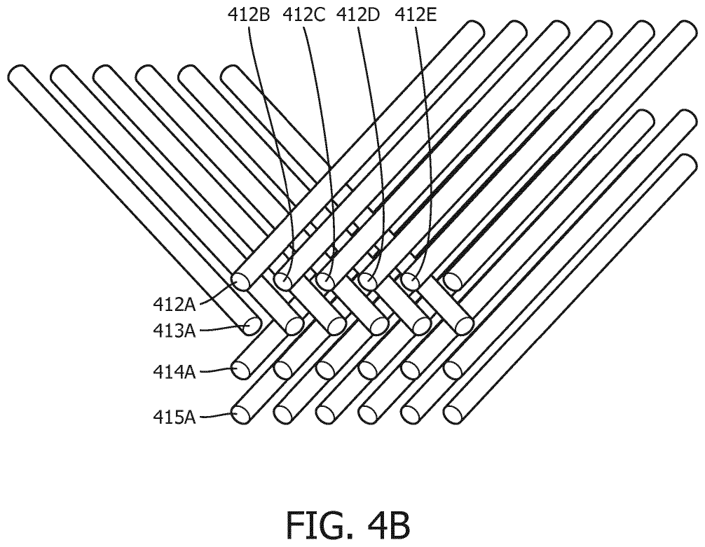

Reference is now made to FIG. 4A, which is a simplified illustration of conducting fibers arranged in layers.

FIG. 4A shows a first layer of conducting fibers 402A 402B 402C 402D 402E; a second layer of conducting fibers represented for the sake of simplicity by one fiber 403A of the second layer; a third layer of conducting fibers represented for the sake of simplicity by one fiber 404A of the third layer; and a fourth layer of conducting fibers represented for the sake of simplicity by one fiber 405A of the fourth layer.

FIG. 4A illustrates, by way of a non-limiting example, muscle fibers, arranged in layers.

Reference is now made to FIG. 4B, which is a simplified illustration of conducting fibers arranged in layers.

FIG. 4B shows a first layer of conducting fibers 412A 412B 412C 412D 412E aligned in a first direction; a second layer of conducting fibers, represented for the sake of simplicity by one fiber 413A of the second layer, aligned in another direction; a third layer of conducting fibers, represented for the sake of simplicity by one fiber 414A of the third layer, aligned in another direction; and a fourth layer of conducting fibers represented for the sake of simplicity by one fiber 415A of the fourth layer, aligned in a direction parallel to the third layer.

FIGS. 4A-B illustrate, by way of a non-limiting example, longitudinal cells or muscle fibers, arranged in layers, with some layers being aligned in a different directions than others.

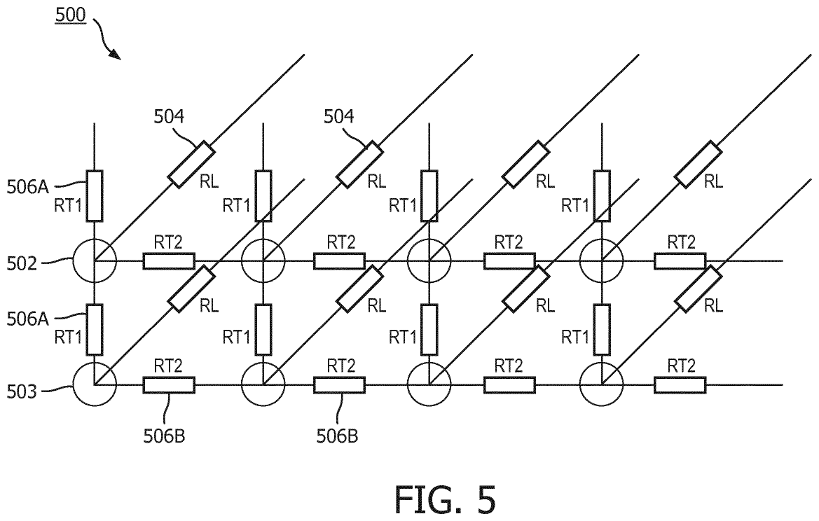

Reference is now made to FIG. 5, which is a simplified illustration of a model of resistance along and between conducting fibers, according to exemplary embodiments of the invention.

FIG. 5 shows a model 500 of resistance including a first layer of conducting fibers 502 (only one fiber is referenced, for simplicity of presentation); a second layer of conducting fibers 503 (only one fiber is referenced, for simplicity of presentation); and resistors 504 506A 506B between the fibers. The resistors are shown to represent resistance or conductance between fibers 502 503.

In FIG. 5, resistors 504 represent resistance along fibers 502 or 503.

In FIG. 5, resistors 506A represent resistance between fibers 502 503 of different layers.

In FIG. 5, resistors 506B represent resistance between fibers of a same layer.

In some embodiments calculations using the model 500 use same values for the resistors 506A and the resistors 506B.

In some embodiments calculations using model 500 use different values for the resistors 506A and the resistors 506B.

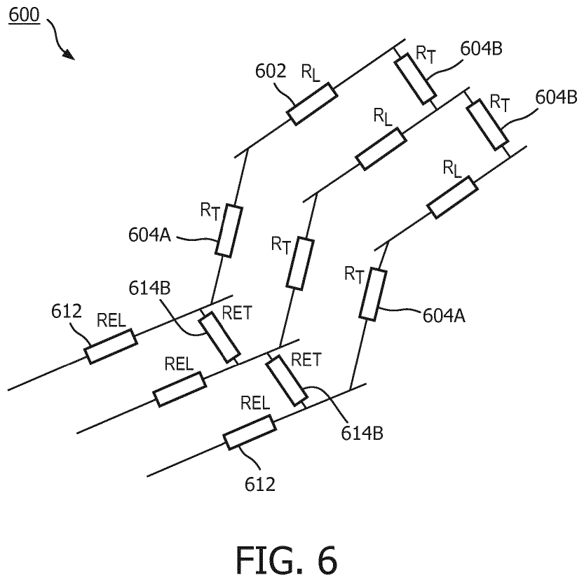

Reference is now made to FIG. 6, which is a simplified illustration of a model of resistance along and between conducting fibers, according to exemplary embodiments of the invention.

FIG. 6 shows a model 600 of resistance including a first layer of conducting fibers represented by longitudinal resistance 602 and transverse resistance 604B and a second layer of conducting fibers represented by longitudinal resistance 612 and transverse resistance 614B.

FIG. 6 also shows the model 600 includes resistance between the layers referenced by 604A.

In some embodiments calculations using the model 600 use same values for the resistors 604A and the resistors 604B. In some embodiments calculations using model 600 use different values for the resistors 604A and the resistors 604B.

In some embodiments, measuring and/or determining multiple tissue impedances and calculating an inner layer's tissue impedance is optionally performed as follows:

Tissue impedance is measured between electrode locations, providing first conductance longitudinal impedance R_{L}, and transverse impedance R_{T} values, corresponding to a first model having one first conductive layer. In some embodiments the value of the longitudinal impedance R_{L}, is selected to be the lowest directional impedance, and the transverse impedance R_{T} is selected to be the highest directional impedance;

a second iteration of the calculation is performed with a second model which includes a second conductive layer which is deeper than the first conductive layer. In the second model, the second conductive layer is connected to the first conductive layer by an impedance value which is similar to the impedance value of the transverse impedance R_{T} of the first layer from the first model.

In some embodiments the deeper layer impedance (in this example: the second layer) is optionally calculated by adjusting the second model for superficial impedance recording.

In some embodiments the above steps are repeated for additional layers, using models with additional layers, potentially providing directional conductance of deeper tissue layers.

In some embodiments conductance of different layers is optionally determined by transmitting signals of different frequencies, which potentially pass through different layers with different impedances, potentially enabling to determine separate impedance values for the layers.

In some embodiments, conductance isotropy is calculated, optionally by providing values representing a difference or a ratio between conductances of different directions at a same location.

In some embodiments, conductance isotropy is calculated, optionally by providing values representing a difference or a ratio between R_{L} and R_{T} at a same location.

An aspect of some embodiments of the invention relates to utilization of a conductance isotropy imaging.

In some embodiments, a likelihood of re-entry arrhythmia formation is optionally calculated, based in part of a change in conductance isotropy values.

A non-limiting example embodiment of such a calculation is described below:

Taking a Gaussian distribution of conduction velocities V_{L} and V_{T}, where V_{L} is a longitudinal conduction velocity and V_{T} is a transverse conduction velocity.

Noting the distribution as: V_{L}+/−E; V_{T}+/−E.

Taking E to be a function F of the conduction isotropy:

E=F(Isotropy) Equation 1

where Isotropy is defined as a ratio V_{L}/V_{T}.

In some embodiments the conduction speed is optionally defined with a value of 1 in a longitudinal direction, that is, V_{L}=1, and so by definition V_{T}=1/Isotropy.

A probability of having multiple parallel pathways each with a specific V_{L} speed is optionally calculated such that a set of conduction velocities is produced for multiple parallel adjacent pathways with an ordered set of speeds for a unit of time (T)—causing for example an angular rotation of the propagation front by 20 degrees. In some embodiments, the multiple parallel adjacent pathways includes at least 5 parallel adjacent pathways. In some embodiments the number of multiple parallel contiguous pathways includes 2, 3, 4, 5, 6, 7, higher numbers up to 20, and even higher numbers up to 100, 500, 1000.

In some embodiments a probability of having N sequential ordered sets of the above velocities is calculated.

In some embodiments a probability of having N=9 sequential ordered sets of the above velocities is calculated.

In some embodiments a requirement is added to the calculation model, that an average V_{L} relate to a refractory period (RP) such that:

RP<a*T

Where “a” is a specific multiplication factor, for example in some embodiments a=10; and T is time.

Such a sequence of ordered sets can potentially cause re-entry arrhythmia.

In some embodiments other ordered set conditions that can cause re-entry arrhythmia are optionally calculated.

In some embodiments a sum of the probabilities is optionally calculated as:

P_{re-entry}=F(Isotropy)

In some embodiments a conductance isotropy mapping is optionally imaged and optionally displayed.

In some embodiments the conductance isotropy mapping is optionally color coded.

In some embodiments the conductance isotropy mapping is optionally used to generate a P_{re-entry} image and/or map.

Some example embodiments are now additionally described:

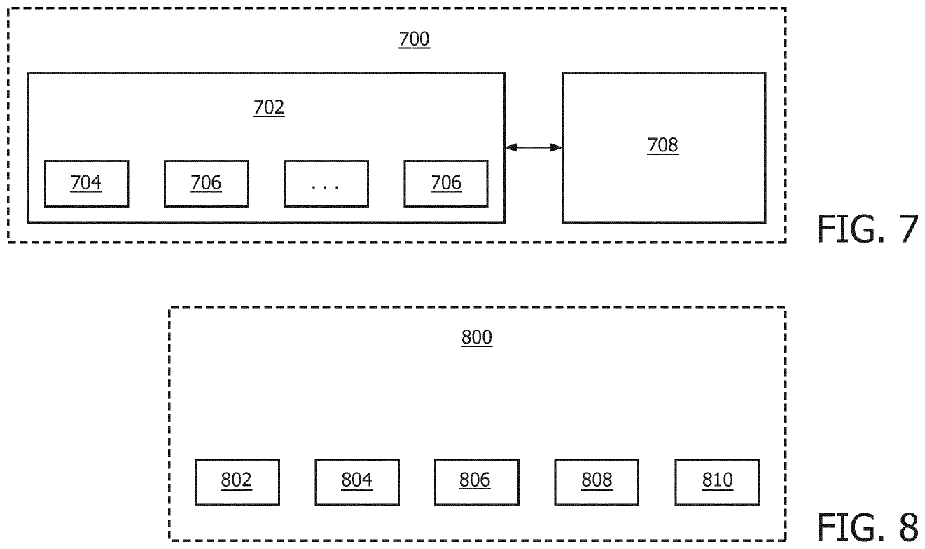

Reference is now made to FIG. 7, which is a simplified block diagram of a system 700 according to some embodiments of the invention.

FIG. 7 shows a system 700 for measuring and/or calculating and/or displaying tissue conductance isotropy including. System 700 may include a catheter 702 which includes a current source electrode 704; a plurality of induced voltage measuring electrodes 706.

Catheter 702 may be, in some embodiments, identical or substantially identical to catheter 206 of FIG. 2 or catheter 304 of FIG. 3.

System 700 may include a signal processing unit 708 for calculating tissue conductance isotropy based on tissue conductance measured in a first direction and tissue conductance measured in a second direction.

Reference is now made to FIG. 8, which is a simplified block diagram of a system 800 according to some embodiments of the invention.

FIG. 8 shows a system 800 for measuring and/or calculating tissue conductance isotropy including one or more of:

contact signal transmitting means 802;

contact signal receiving means 804;

remote signal receiving means 806;

a signal processing unit 808; and

a display unit 810.

In some embodiments, signal processing unit 808 is configured to calculate impedance between the contact transmitting and receiving means, adjusting for transmitting the impedance to the receiving means.

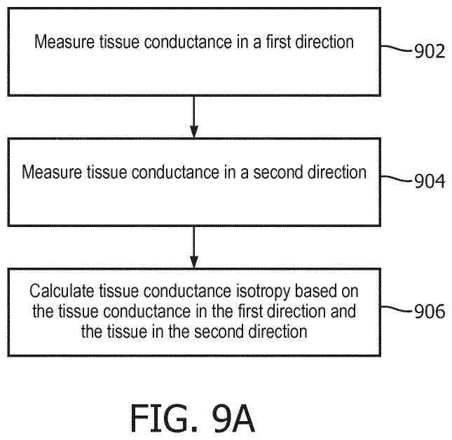

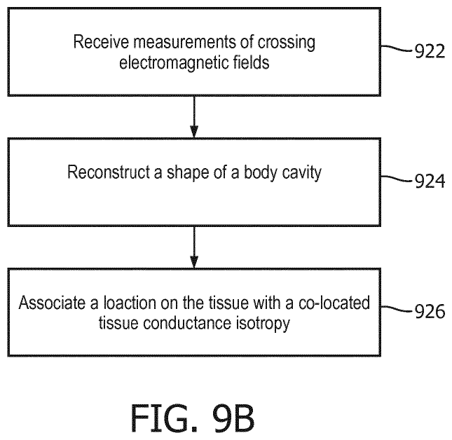

Reference is now made to FIG. 9A, which is a simplified flow chart illustration of a method for measuring tissue conductance isotropy according to some embodiments of the invention.

The method of FIG. 9A includes one or more steps of:

measuring tissue conductance in a first direction (902);

measuring tissue conductance in a second direction (904); and

calculating tissue conductance isotropy based on the tissue conductance in the first direction and the tissue conductance in the second direction (906), wherein the second direction is not parallel to the first direction.

Measuring tissue conductance may be according to any one of the methods described above.

Reference is now made to FIG. 9B, which is a simplified flow chart illustration of a method for mapping tissue conductance isotropy according to some embodiments of the invention.

The method of FIG. 9B includes one or more steps of:

receiving measurements of crossing electromagnetic fields (922), optionally using two sensors carried on an intra-body catheter at optionally a known distance from each other, the measuring being carried out with the catheter at multiple locations in the body cavity; and

reconstructing a shape of the body cavity (924), optionally based on the received measurements, reconstruction may be in accordance with methods described in the above mentioned PCT Patent Application IB 2018/050192;

receiving or calculating tissue conductance isotropy based on the received measurements, for example: according to any one of the methods described above; and

associating a location on tissue with a co-located tissue conductance isotropy (926), e.g., to obtain a map or image of the tissue conductance isotropy.

The method may further include displaying or otherwise providing to a user such map or image of the tissue conductance isotropy.

In some embodiments, the plurality of crossing electromagnetic fields include at least one electromagnetic field established between electrodes of the sensors.

In some embodiments, crossed or crossing fields are fields directed in directions that are not parallel to each other, nor anti-parallel, so that the direction of each field crosses the directions of all the other fields.

It is expected that during the life of a patent maturing from this application many relevant methods of measuring electric conductance of tissue will be developed and the scope of the term measuring electric conductance in all its grammatical forms is intended to include all such new technologies a priori.

As used herein with reference to quantity or value, the term “about” means “within ±25% of”.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for measuring tissue conductance isotropy comprising:
measuring tissue conductance in a first direction, using a current source and a measuring electrode provided on a same catheter or on a same electrode implanted in tissue;
measuring tissue conductance in a second direction, using the current source and a measuring electrode provided on the same catheter or on a same electrode implanted in tissue; and
calculating tissue conductance isotropy based on the tissue conductance in the first direction and the tissue conductance in the second direction, wherein the second direction is not parallel to the first direction.

2. The method of claim 1 wherein values of tissue conductance in the first direction and in the second direction are calculated as tissue conductance in a longitudinal direction C_{L} and in a perpendicular transverse direction C_{T}.

3. The method of claim 2 wherein the values of tissue conductance in a longitudinal direction C_{L} is determined in a direction of maximum conductance.

4. The method of claim 2 wherein the values of tissue conductance in a transverse direction C_{T} is determined in a direction of minimum conductance.

5. (canceled)

6. (canceled)

7. The method of claim 1 wherein the measuring tissue conductance in the first direction and the measuring tissue conductance in the second direction are performed simultaneously.

8. The method of claim 1 wherein the current source is provided by an electrode implanted in tissue.

9. The method of claim 8 wherein the measuring tissue conductance in the first direction and the measuring tissue conductance in the second direction is performed by a measuring electrode on a same implanted electrode as the current source.

10. The method of claim 8 wherein the measuring tissue conductance in the first direction and the measuring tissue conductance in the second direction is performed by a measuring electrode on a same implanted electrode as the current source.

11. The method of claim 1 wherein the current source is provided by an electrode on a catheter.

12. The method of claim 1 wherein the measuring tissue conductance in the first direction is performed by a measuring electrode provided on a catheter.

13. The method of claim 11 wherein the measuring tissue conductance in the second direction is performed by a measuring electrode provided on a same catheter as the current source.

14. (canceled)

15. The method of claim 11 wherein the catheter is placed next to the tissue being measured.

16. The method of claim 11 wherein the catheter is within a body cavity during the measurement.

17. The method of claim 11 wherein the catheter is within a blood vessel during the measurement.

18. The method of claim 11 wherein the catheter is within a heart during the measurement.

19. The method of claim 1 wherein tissue conductance is measured in more than two directions at a same source location.

20. The method of claim 1 wherein tissue conductance is measured in more than two directions simultaneously.

21. The method of claim 11 wherein:
the catheter is translated along the tissue;
additional conductance measurements are performed; and
further comprising providing locations of the measurements.

22. The method of claim 21 wherein same electrodes are used to measure conductance and to provide data for providing the locations.

23. The method of claim 21 and further comprising producing a map of tissue conductance isotropy based, at least in part, on the locations.

24. The method of claim 23 wherein the map is selected from a group consisting of:
a one-dimensional map;
a two-dimensional map; and
a three-dimensional map.

25. The method of claim 23 wherein the map displays different tissue conductance isotropy using different colors.

26. The method of claim 1 wherein the calculating tissue conductance isotropy is performed for a same location at different times, and a change in tissue conductance isotropy is calculated.

27. The method of claim 1 wherein the calculating tissue conductance isotropy is performed at different times during one cardiac cycle.

28. The method of claim 1 wherein the tissue conductance isotropy is combined with ECG data.

29. The method of claim 23 wherein the map is produced for a same location at different times, and a map of change in tissue conductance isotropy is calculated.

30. The method of claim 29 wherein the map of change is displayed in color based on an amount of change.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. A system for measuring tissue conductance isotropy comprising:
means for measuring tissue conductance in a first direction, using a current source and a measuring electrode provided on a same catheter or on a same electrode implanted in tissue;
means for measuring tissue conductance in a second direction, using the current source and a measuring electrode provided on the same catheter or on a same electrode implanted in tissue; and
means for calculating tissue conductance isotropy based on the tissue conductance in the first direction and the tissue conductance in the second direction.

49. A computer program product comprising a computer readable medium having computer readable code embodied therein, the computer readable code being configured such that, on execution by a suitable computer or processor, the computer or processor is caused to perform the method of claim 1.
